(19) United States
(12) Patent Application Publication
(10) Pub. No.: US 2009/0144872 A1
(43) Pub. Date: Jun. 11, 2009

(54) Attachment System For A Helmet

(76) Inventors: Stephane Lebel, St.-Redempteur (CA); Martin Belanger, Quebec City (CA); Pierre Vallee, Quebec City (CA)

Correspondence Address: THE LAW OFFICE OF RANDALL T. ERICKSON, P.C., 1749 S. NAPERVILLE ROAD, SUITE 202, WHEATON, IL, 60187, US

(21) Appl. No.: 12/202,212

(22) Filed: Aug. 29, 2008

Related U.S. Application Data
(60) Provisional application No. 60/966,645, filed on Aug. 29, 2007.
(60) Provisional application No. 60/966,817, filed on Aug. 30, 2007.

(51) Int. Cl. F41H 1/04; A42B 1/24; A42B 1/08; A61F 9/00; A41D 13/00

(52) U.S. Cl. 2/6.7; 2/422; 2/424; 2/10; 2/468

(57) ABSTRACT

An attachment system for attaching a face shield to a helmet. The system comprises a pair of attachment bases for holding a face shield to the helmet and for attaching to opposite sides of the helmet. The attachment bases each include a base plate covered by a body block. The base plate includes hooks for engaging an edge of the helmet. A strap assembly provides a pair of tightening straps, each engaging one attachment base. The tightening straps extend rearward and are connected together by a helmet band that extends over and around a portion of the helmet. The tightening straps each include a ratchet toothed strap body for adjusting a tension of the straps against the helmet.

This application claims the benefit of U.S. Provisional Patent Application Nos. 60/966,645, filed Aug. 29, 2007 and 60/966,817, filed Aug. 30, 2007.

### TECHNICAL FIELD OF THE INVENTION

The present invention relates to attachment systems for use with helmets, and particularly for impact resistant face shields for use in military helmets and in law-enforcement helmets.

### BACKGROUND OF THE INVENTION

Face shields for use in military and law-enforcement helmets are known such as disclosed in U.S. Pat. Nos. 5,901,369 and 4,536,892. These face shields provide pivot arrangements on opposite lateral sides of the face shield to allow the face shield to be pivoted upward away from the user's face when the face shield is not deployed. The face shield is held in a deployed position in front of the user's face by locking of the pivot arrangements.

Also, face shields are used with industrial safety helmets, motorcycle helmets, winter sport helmets and auto racing helmets.

The present inventors have recognized that it would be desirable to provide a helmet attachment system for attaching a face shield that was compatible with different style helmets with minimum reconfiguration required.

Furthermore, the present inventors have recognized that it would be desirable to provide an attachment system that was usable to attach other devices.

The present inventors have also recognized that it would be desirable to provide an attachment system that was quickly and easily installed onto or removed from a helmet.

### SUMMARY OF THE INVENTION

The invention provides a helmet attachment system for attaching a face shield that is compatible with different style helmets with a minimum reconfiguration required. The present invention provides a helmet attachment system that is usable to attach other devices in addition to the face shield. The present invention provides an attachment system that is quickly and easily installed onto or removed from a helmet.

The helmet attachment system of the present invention comprises a pair of attachment bases, each arranged on a side of the helmet near to an ear covering of the helmet. The attachment bases each include a metal base plate covered by an attached body block. Each attachment base provides provisions for attaching devices to the attachment base, such as a mandible, a camera and/or a flashlight. The metal base plate includes hooks for engaging an edge of the helmet. A strap assembly provides a pair of tightening straps, each engaging one attachment base. The tightening straps extend rearward and are connected together by a helmet band that extends over and around the back of the helmet.

The tightening straps each include a ratchet toothed strap body that is connected to the helmet band by a latch mechanism wherein a selected tooth along a length of the toothed body can be engaged by the latching mechanism. The tightening straps are each connected to an attachment base by a strap tension locking mechanism that has an unlocked position having an extended length and a folded, locked position having a reduced length to exert tension on the tightening straps.

The body block of the attachment base can be fastened to either of two metal base plates that are each specifically configured to be compatible with the contours of a specific military helmet. In this way the body block of the attachment base can be a universal part.

The attachment bases and the face shield provide an effective, cost efficient manner of holding the faced shield in either the downward tilted, deployed position or the upwardly tilted, non-use position. The face shield has two recesses located near to the pivot axis of the face shield, rotationally spaced apart and corresponding to the two rotational positions of the face shield, the downward tilted, deployed position or the upwardly tilted, non-use position. The attachment base includes a resilient detent that fits into the selected recess and holds the rotational position of the face shield, but can be moved into and out of the selected recess to change the rotational position of the face shield by a firm pivoting force on the face shield by the user.

Numerous other advantages and features of the present invention will be become readily apparent from the following detailed description of the invention and the embodiments thereof, and from the accompanying drawings.

### BRIEF DESCRIPTION OF THE DRAWINGS

FIG. 1 is a perspective view of a helmet outfitted with the attachment system of the present invention;

FIG. 2 is an enlarged, fragmentary, perspective view of the helmet outfitted with the attachment system shown in FIG. 1;

FIG. 3 is a front perspective view of the a helmet and mandible outfitted with the attachment system of the present invention;

FIG. 4 is an enlarged fragmentary side view of the helmet and mandible outfitted with the attachment system taken from FIG. 3 shown with the face shield tilted up;

FIG. 5 is an enlarged fragmentary side view of the helmet and mandible outfitted with the attachment system taken from FIG. 3 shown with the face shield tilted down;

FIG. 6 is an enlarged perspective view of one attachment base taken from FIG. 3;

FIG. 7 is an enlarged fragmentary outside side view of the attachment base mounted to the helmet taken from FIG. 3;

FIG. 8 is an enlarged fragmentary perspective view taken from FIG. 3 with the attachment base removed to view obscured parts;

FIG. 9 is an enlarged fragmentary perspective view taken from FIG. 3 with a strap removed to view obscured parts;

FIG. 10 is an enlarged fragmentary inside perspective view of the helmet and mandible with the attachment system of the invention taken from FIG. 3;

FIG. 11 is an enlarged fragmentary top perspective view taken from FIG. 7 with the face shied removed to reveal obscured parts;

FIG. 12 is an enlarged fragmentary side view of the attachment system of the invention taken from FIG. 3 showing the attachment system unlocked;

FIG. 13 is a helmet-side view of the attachment base;

FIG. 14 is a helmet-side view of the body block of the attachment base, with a metal base plate and metal hardware removed;

FIG. 15 is a helmet side view of the metal base plate and metal hardware of the attachment base with the body block removed;

FIG. 16 is a outside side view of the metal base plate and metal hardware of the attachment base with the body block removed;

FIG. 17 is a helmet-side view of an alternate attachment base using a different base plate;

FIG. 18 is an enlarged fragmentary inside perspective view of a helmet with the attachment system of the invention taken from FIG. 17;

FIG. 19 is an enlarged fragmentary rear perspective view of a portion of the helmet outfitted with the attachment system of FIG. 1;

FIG. 20 is a right side perspective view of an alternate helmet attachment system of the invention;

FIG. 21 is a right side perspective view of the alternate helmet attachment system of FIG. 20 with a visor removed to view underlying parts;

FIG. 22 is a right side perspective view of the alternate helmet attachment system of FIG. 21 with a portion removed to view underlying parts;

FIG. 23 is a right side perspective view of a further alternate helmet attachment system with a portion removed to view underlying parts;

FIG. 24 is a right side perspective view of the alternate helmet attachment system of FIG. 23 with portions removed to view underlying parts;

FIG. 25 is a right side perspective view all the further alternate helmet attachment system of FIG. 24 with portions removed to view underlying parts;

FIG. 26 is a perspective view of a portion of the alternate and further alternate helmet attachment systems of FIGS. 20 and 23;

FIG. 27 is a sectional view taken generally along line 27-27 of FIG. 22 and FIG. 23;

FIG. 28 is a right side perspective view of a portion of the alternate helmet attachment system of either FIG. 20 or FIG. 23;

FIG. 29 is a left side perspective view of the portion shown in FIG. 28;

FIG. 30 is a perspective view of attachment portions shown in FIGS. 22 and 23;

FIG. 31 is a top view of an attachment portion with other portions removed for viewing underlying parts;

FIG. 32 is a perspective view of the attachment portion shown in FIG. 31;

FIG. 33 is a right side perspective view of the attachment portion shown in FIG. 32 with a portion removed to view underlying parts;

FIG. 34 is a right side perspective view of the attachment portion shown in FIG. 33 with a portion removed to view underlying parts; and

FIG. 35 is a front view of the attachment portion shown in FIG. 34 with a portion removed to view underlying parts.

### DETAILED DESCRIPTION OF THE PREFERRED EMBODIMENTS

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

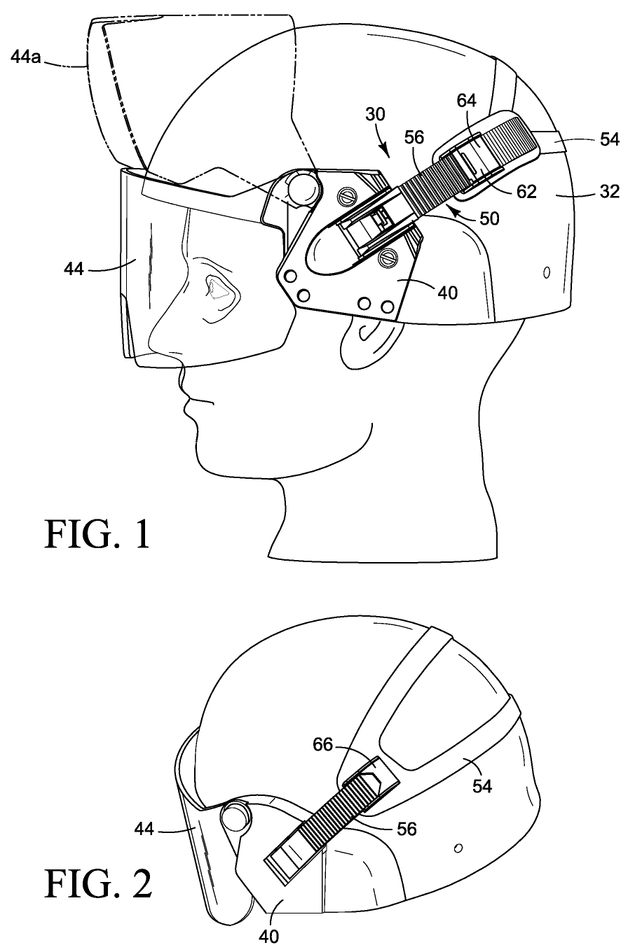

FIGS. 1 and 2 illustrate an attachment system 30 according to the present invention mounted to a military helmet 32. The attachment system includes left and right attachment bases (left and right orientations mentioned herein are according to the helmet wearer's perspective). For simplicity, only the left attachment base 40 is shown with the understanding that the right attachment base would be mirror image identical across a vertical center plane through the helmet between the wearer's eyes.

A face shield 44 can be pivotally mounted to the left (and right) attachment base 40 to be movable between a deployed, tilted down position shown in FIG. 1 or a tilted up position indicated as 44a. The face shield can be made of polycarbonate or urethane or other transparent material. For military use particularly, it should be ballistic impact resistant. It can be of a laminated construction and can feature a variable light transmission system. Such a variable light transmission system can incorporate an electronic control system to vary the light transmission according to the ambient light conditions. For firefighting applications the face shield should be of a heat resistant material.

The attachment base 40 engages the helmet as described below and is drawn rearward by a strap system 50. Only the left side of the strap system 50 is shown in FIG. 1 but it is to be understood that the right side would be mirror image identical across a vertical center plane through the helmet between the wearer's eyes. The strap system includes an oval-shaped helmet band 54 that braces against a rear surface of the helmet 32. The oval-shaped helmet band provides reduced slipping of the band on the helmet and greater stability of the attachment bases. The strap system includes left and right tightening straps. Only the left tightening strap 56 is shown with the understanding that the right tightening strap would be mirror image identical across a vertical center plane through the helmet between the wearer's eyes. Left and right strap-engaging clasps are mounted on opposite ends of the band 54. Only the left strap-engaging clasp 62 is shown with the understanding that the right strap-engaging clasp would be mirror image identical across a vertical center plane through the helmet between the wearer's eyes.

The left strap-engaging clasp 62 includes a pivotal latch 64 (FIG. 1) and a base 66 (FIG. 2). The latch 64 is removed in FIG. 2 to reveal the base 66. The strap 56 includes an elongated strap body 67 having ratchet angled teeth 68. In operation, the latch 64 is pivoted away from the base 66 and the strap body 67 is inserted between the latch 64 and the base 66 to a selected extent. The latch is released and it engages a ratchet tooth 68 to fix the strap body 67 in positioning within the clasp 62. The left strap-engaging clasp 62 is not described in great detail herein with the understanding that clasps of this nature are well known and in wide use to engage ski boot tightening straps and snowboard boot binding straps. The clasp can be a manual clasp or can have a ratchet lever to pull the strap body through the clasp 62 to exert a tightening force on the strap body 67. U.S. Pat. Nos. 5,416,952; 5,887,318; 3,662,435; 5,909,850; 4,667,424; 5,606,779; 6,175,994; 5,606,779; and 5,779,259 all describe various toothed straps and clasps, and they are all herein incorporated by reference.

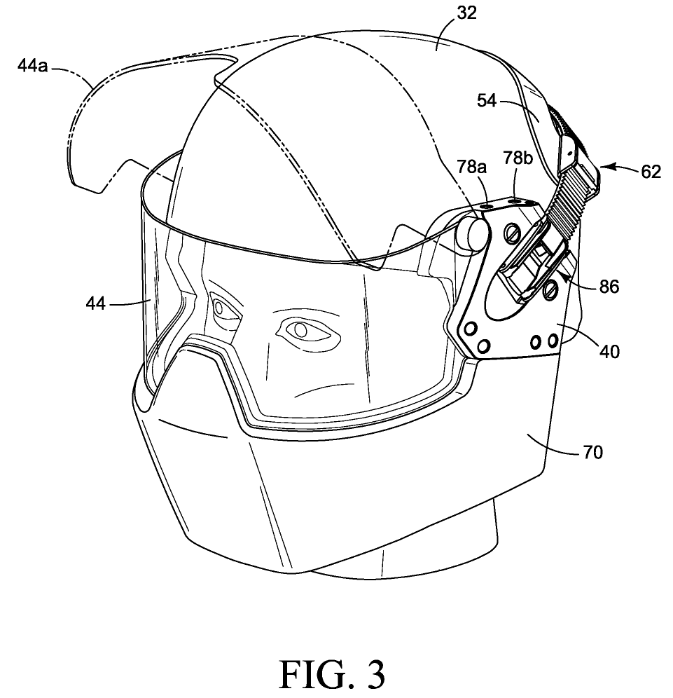

FIG. 3 illustrates a mandible or mouth guard 70 that is attached to the helmet 32 and fits in close conformity to the attachment bases. Only the fit to the left attachment base 40 is shown with the understanding that the fit to the right attachment base would be mirror image identical across a vertical center plane through the helmet between the wearer's eyes.

For military applications particularly, the mandible should be ballistic impact resistant. The mandible could advantageously be used for motorcycle helmet, winter sport or auto racing applications. The mandible should have a high mass impact resistance.

The attachment base 40 includes three pair of attachment holes 74a, 74b; 76a, 76b; and 78a, 78b. These attachment holes are for the attachment of auxiliary equipment to the helmet such as a flashlight, camera or other tools or devices, or to attach a PICATINY rail to adapt standard component attachments such as for a flashlight or camera. The attachment, holes can be used to attach the mandible 70 to the attachment base 40 to mount the mandible 70 to the helmet 32. The holes 74a, 74b; 76a, 76b; and 78a, 78b can be plain or threaded.

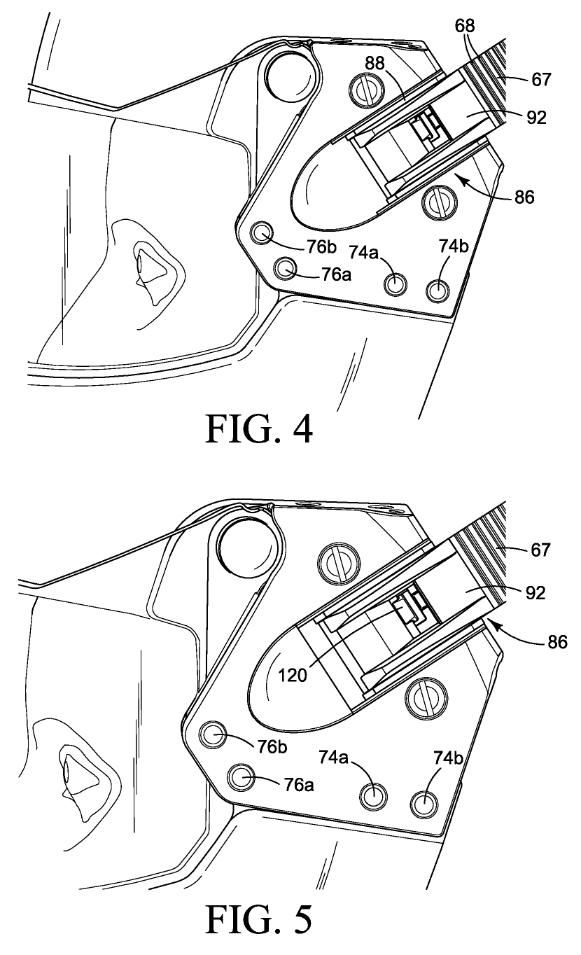

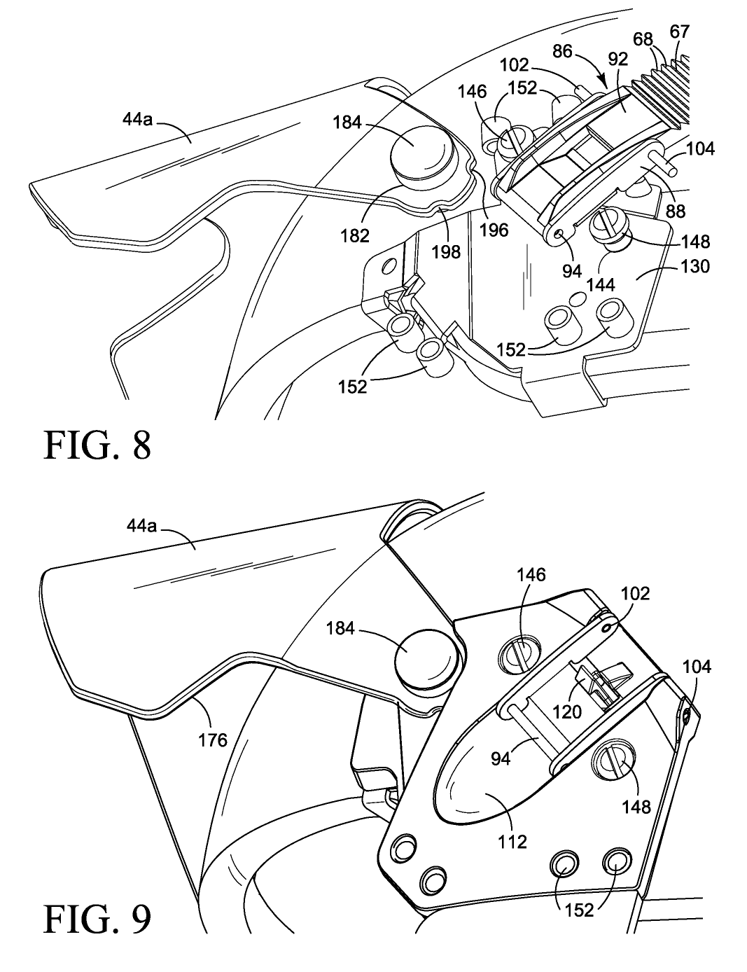

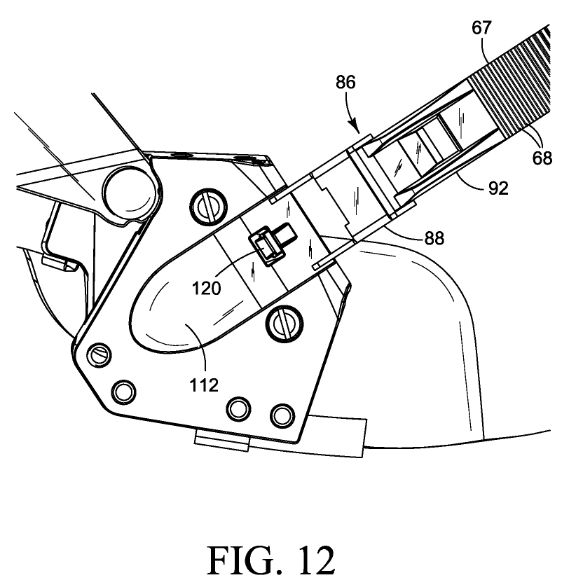

FIGS. 4-9 illustrate the strap 56 includes a strap tension lock 86. The tension lock 86 includes an H-shaped base 88 that is pivotally connected to a strap head 92 by a pin 94. The base is pivotally connected to the attachment base 40 by two pins 102, 104. The strap head is connected to, or formed with a toothed strap body 67. The toothed strap body 67 is selectively engaged along its length by the clasp 62. The base 88 and the strap head 92 can be folded together as shown in FIG. 8 to have the shortest overall combined length, or pivot away from each other as shown in FIG. 12, to assume the greatest overall length and, in effect, release the attachment base from tension.

When the base 88 and the strap head 92 are in the folded position, the tension lock 86 fits within a recess 112 in the attachment base 40. The attachment base 40 includes a latch or tang 120 that extends outwardly through an opening in the strap head 92 to hold the strap head 92 onto the base 88 to retain the base 88 and the strap head 92 in the folded condition to prevent the attachment base 40 from inadvertently loosening and releasing the attachment base.

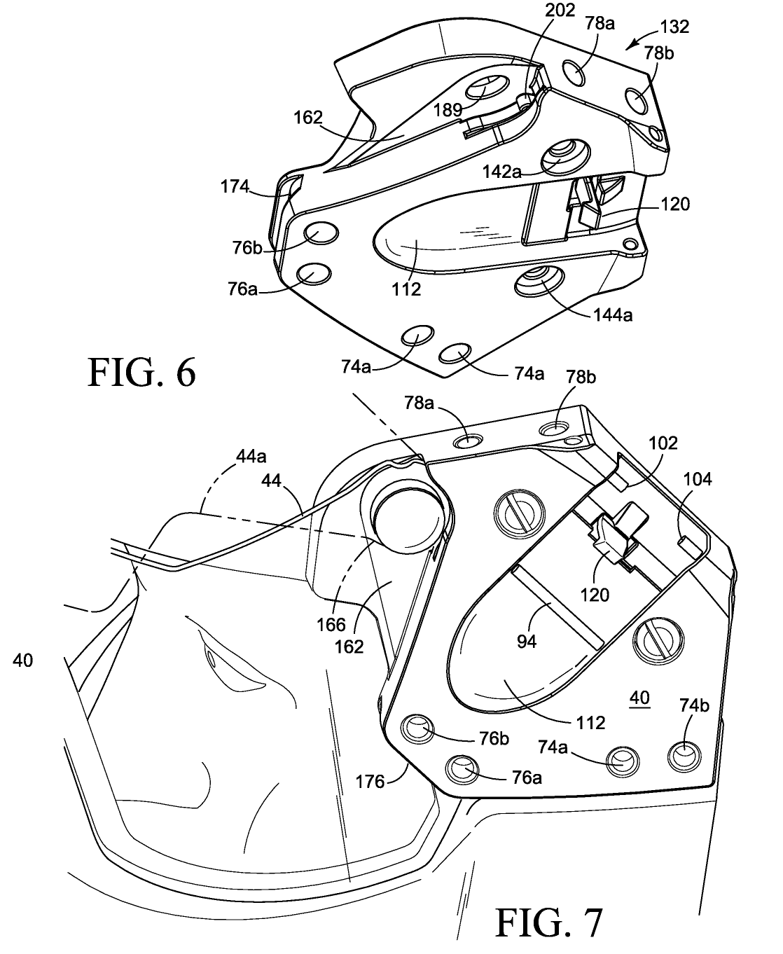

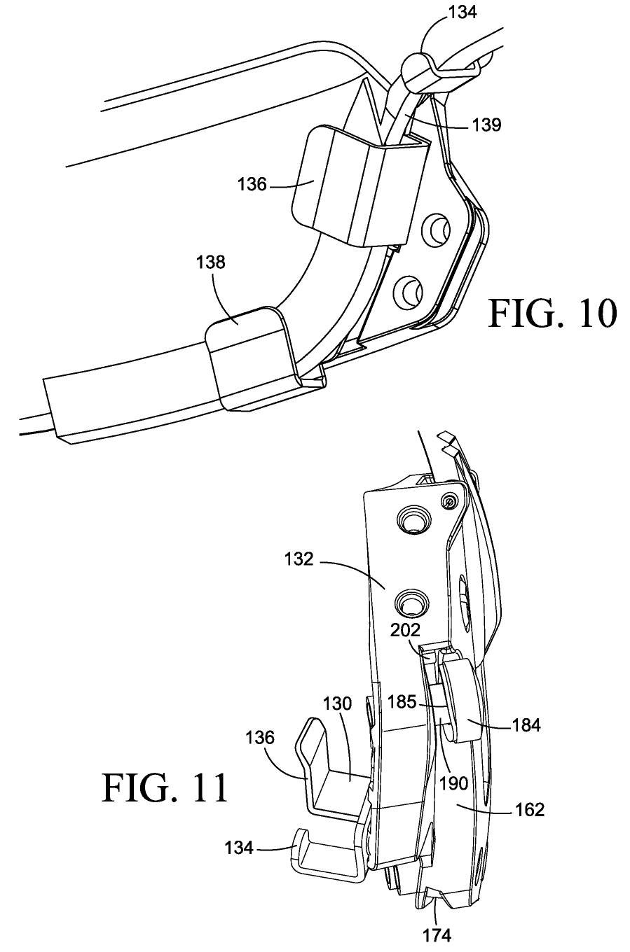

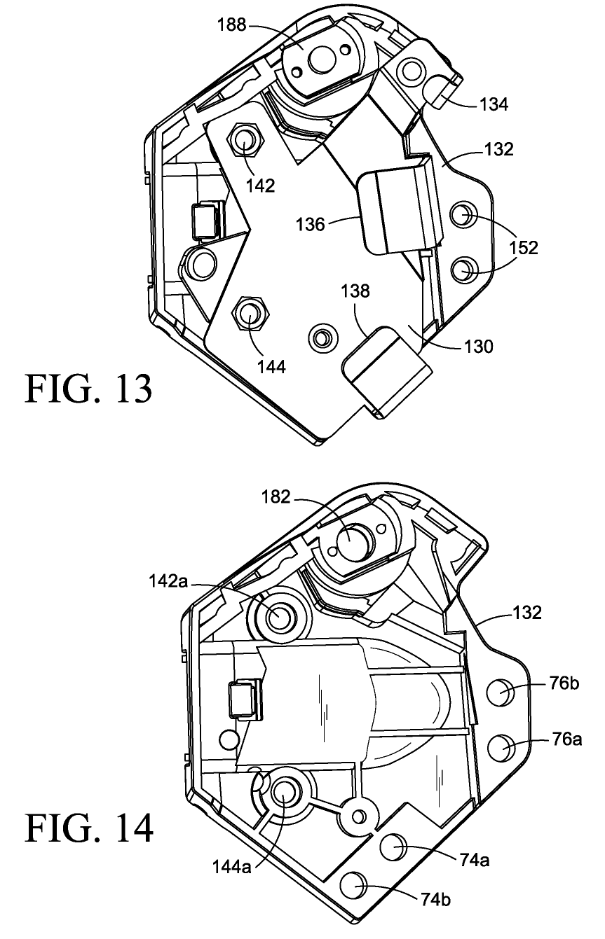

As illustrated in FIGS. 6, 10, 11 and 13-16, the attachment base comprises a base plate 130 and an overlying body block 132. The base plate 130 is preferably a metal part, such as composed of hot roll, satin coat steel, 16 gauge. The body block 132 is preferably a plastic or polymer part such as nylon which can be the material ZYTEL ST801, black, available from E. I. duPont de Nemours & Co., Wilmington, Del., US.

The base plate 130 includes hooks 134, 136, 138 that engage a tower front edge 139 of the helmet 32. The base plate 130 also includes two threaded bosses 142, 144 that fit into countersunk holes 142a, 144a (FIG. 14) through the body block 132 and receive screws 146, 148 that fasten the base plate 130 to the body block 132. A plurality of metal sleeves 152 are provided, each one fitting into one of the holes 74a, 74b; 76a, 76b; and 78a, 78b to reinforce the holes and/or to provide threads if needed. The sleeves 152 can be insert molded or overmolded with the body block 132.

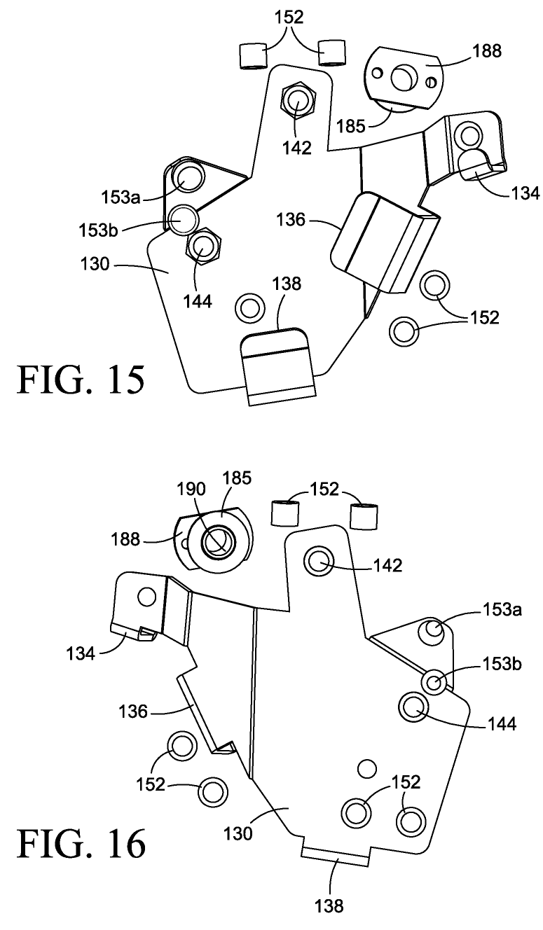

The base plate 130 can be fitted with elastomeric or rubber bumpers or pads 153a, 153b (FIGS. 15 and 16) that can be adhesively secured to the base plate or resiliently pressed into and captured by holes into the base plate as shown. The pads 153a, 153b ensure a stable contact point on the helmet.

The body block 132 provides a recess or mount 162 that receives a side connecting portion 166 of the face shield 44. The body block has a groove 174 that receives a lower side edge 176 of the face shield 44 when the face shield is tilted down to a deployed position. The face shield 44 includes a connection hole 182 at each side for connection to the attachment base 40. A hand-turned screw 184 that penetrates the hole 182 and a spring washer 185, on each side of the shield, attaches the face shield to a mounting anchor 188 that is connected to the body block 132. The mounting anchor 188 can include a threaded boss 190 fit within a hole 189 (FIG. 6) in the body block 132 to receive the hand-turned screw 184. The mounting anchor 188 can be fastened to, captured by, or insert molded with the body block 132.

The face shield 44 can include two recesses 196, 198 that engage a detent 202 (FIGS. 6 and 11) formed into the recess 162. The detent 202 is resilient to allow the detent 202 to enter or escape each recess 196, 198 with a firm force to raise or lower the face shield. Thus, the face shield is held in a selected position by the corresponding recess 196, 198: in the elevated position by the recess 196, or the lowered position by the recess 198.

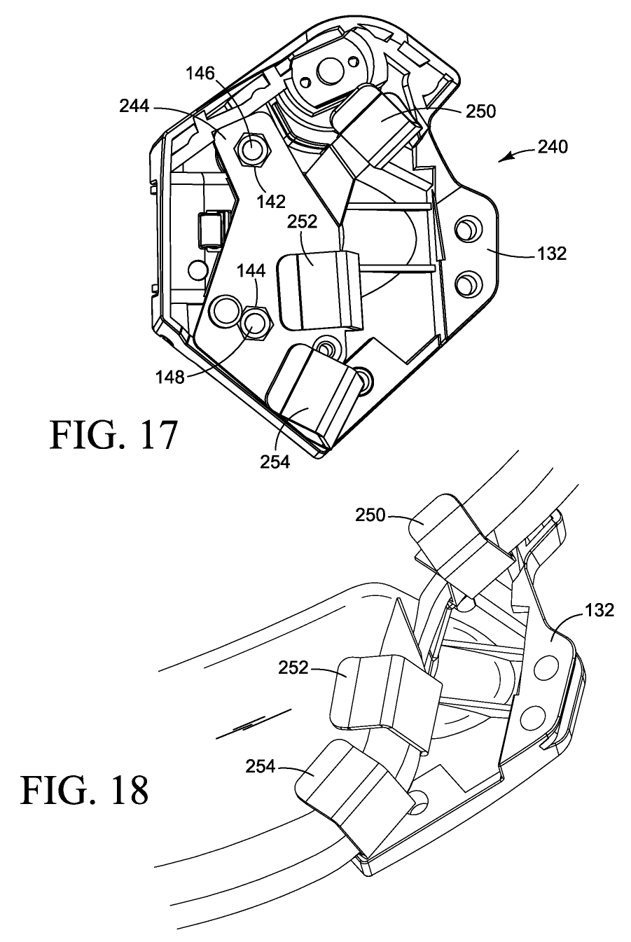

Another advantage of the attachment system is the flexibility to adapt the attachment base 40 fit onto different helmets. FIGS. 17 and 18 illustrate a modified attachment base 240 comprising the body block 132 and a metal base plate 244, different than the metal base plate 130 shown in the previous embodiment. The base plate 244 is of a different shape and has hooks arranged to clasp a different helmet than the helmet 32 shown in FIG. 1. The base plate 244 fastens to the same body block 132 using screws 146, 148 into bosses 142, 144 formed on the base plate 244. In this way the body block 132 can be a universal part for both style helmets.

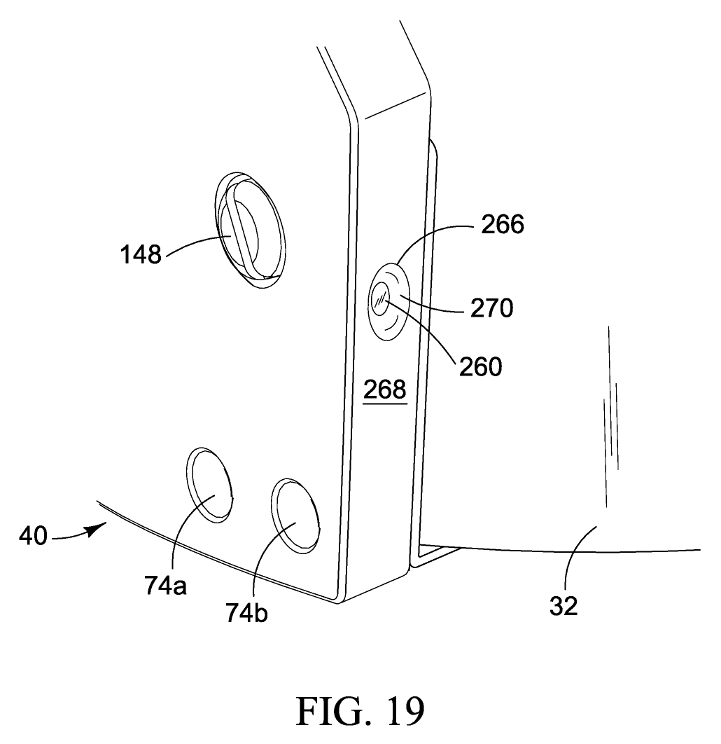

FIG. 19 illustrates a further aspect of the invention. A signal element 260 such as an infrared (IR) light designator (emitter) can be carried by the attachment base 40 or 240. The signal element 260 in the form of an IR light designator is mounted within an opening 266 in a rearward facing surface 268 of the attachment base 40 or 240. The opening can have a cone-like surface 270 with a wide end at the surface 268 to increase the dispersion of the IR signal. More than one signal element 260 can be applied to the attachment base to increase the flexibility or variance of the signal desired to be present on the attachment base. In the case of military use the signal designators can be used to identify the helmet wearer as friend or foe. Where the signal indicator is an IR designator, since it is emitting IR signals, it will be seen at night by other soldiers in the back of the helmet wearer, when the other soldiers wear night vision goggles.

The attachment system 30 can also includes a top edge stop fixture between the helmet and the face shield, or a gasket that extends between the attachment bases, the details of which are disclosed in U.S. patent application Ser. No. 11/811,361, filed Jun. 9, 2007, herein incorporated by reference. Either embodiment resists movement of the face shield in an up and down direction and the gasket also prevents any inflammable liquid from flowing into the helmet. The face shield 44 can also have the thickness profile and properties of the face shield described in U.S. patent application Ser. No. 11/811,361, filed Jun. 9, 2007, herein incorporated by reference.

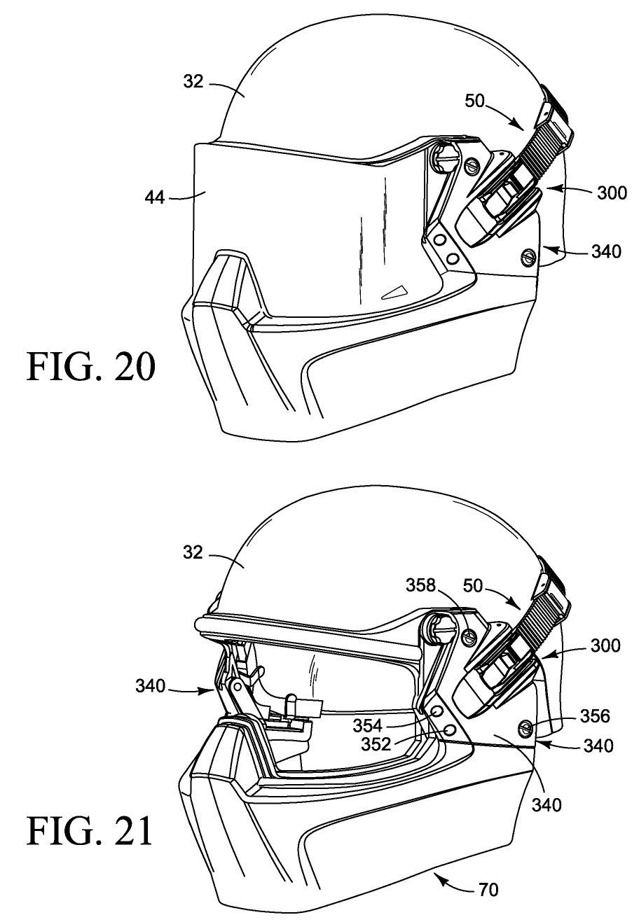

FIGS. 20-21 illustrate an alternate attachment system 300 according to the present invention mounted to a military helmet 32. The attachment system includes left and right attachment bases (left and right orientations mentioned herein are according to the helmet wearer's perspective). For simplicity, only the left attachment base 340 is shown with the understanding that the right attachment base would be mirror image identical across a vertical center plane through the helmet between the wearer's eyes. The attachment base 340 is identical to the above described base 40 except as differentiated herein.

The face shield or visor 44, as described above, can be pivotally mounted to the left (and right) attachment base 340 to be movable between a deployed, tilted down position shown in FIG. 20 or a tilted up position such as shown in FIG. 1 (indicated as 44a).

The attachment base 340 engages the helmet as described below and is drawn rearward by the strap system 50 as described above.

FIG. 21 illustrates a mandible or mouth guard 70 that is attached to the helmet 32 via the attachment bases 340 bases and fits in close conformity to the attachment bases. Only the fit to the left attachment base 340 is shown with the understanding that the fit to the right attachment base would be mirror image identical across a vertical center plane through the helmet between the wearer's eyes.

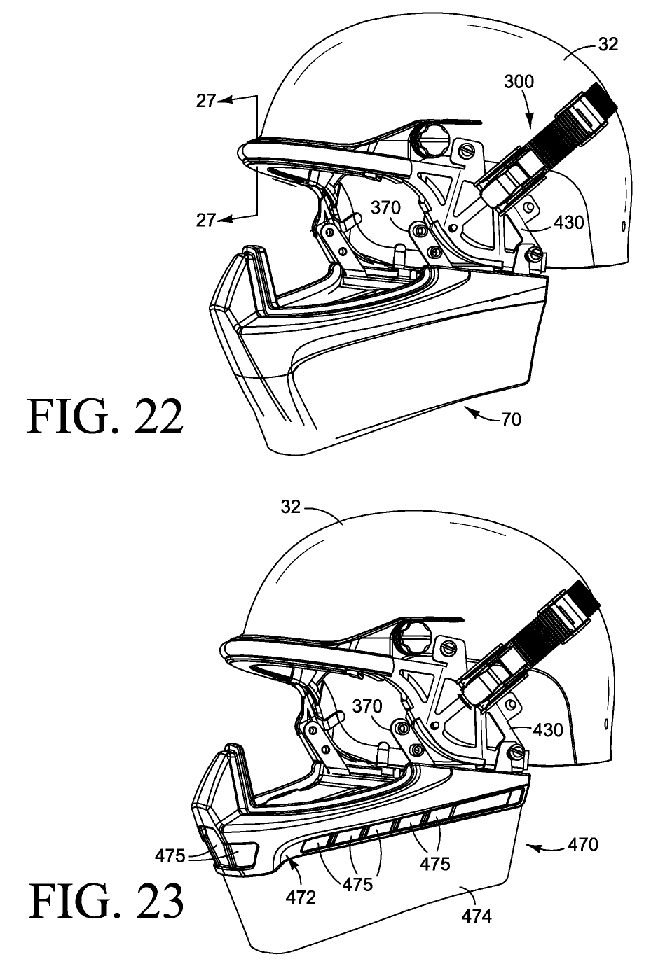

The attachment base 340 includes various attachment holes 352, 354, 356, 358, 360, 362, 364. The attachment holes 352, 354 are arranged for receiving fasteners to attach the mandible to the base 340 via holes 370a, 370b in a mandible bracket 370. The hole 356 is arranged to receive a fastener via a hole 370c in the mandible bracket 370. The mandible bracket includes a perforated base 370c that can be overmolded by the resin of the mandible to fix the bracket 370 to the mandible. The attachment holes 356 and 358 also secure the base plate 430, identical in configuration and function to the prior described base plate 130 except as distinguished herein, to a back side of a body block 432 of the attachment base 340. The base plate 430 has material removed therefrom to reduce weight. The body block 432 is identical to the body block 132 except as distinguished herein. For clarity, the body block is removed in FIGS. 22, 23, 24 and 25 to view underlying parts.

FIG. 23 illustrates a further alternate embodiment identical to the embodiment shown in FIGS. 20-22 except wherein the mandible 70 is replaced by an alternate mandible arrangement 470 that includes a rigid material mandible frame 472 and a ballistic resistant fabric 474 which hangs from the mandible frame 472. The mandible frame 472 has material removed forming openings 475 to reduce weight. The fabric 474 is attached to an inside of the frame 472 such as by adhesive securement to, or overmolding of the frame on the fabric. The fabric covers these openings on the inside of the frame 472, and hangs beneath the frame 472 to protect the neck area of the wearer.

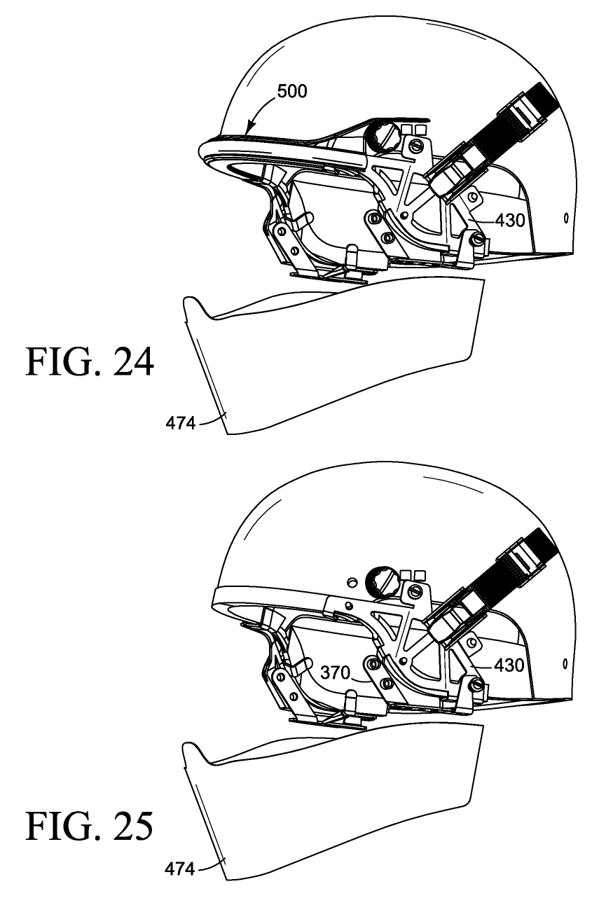

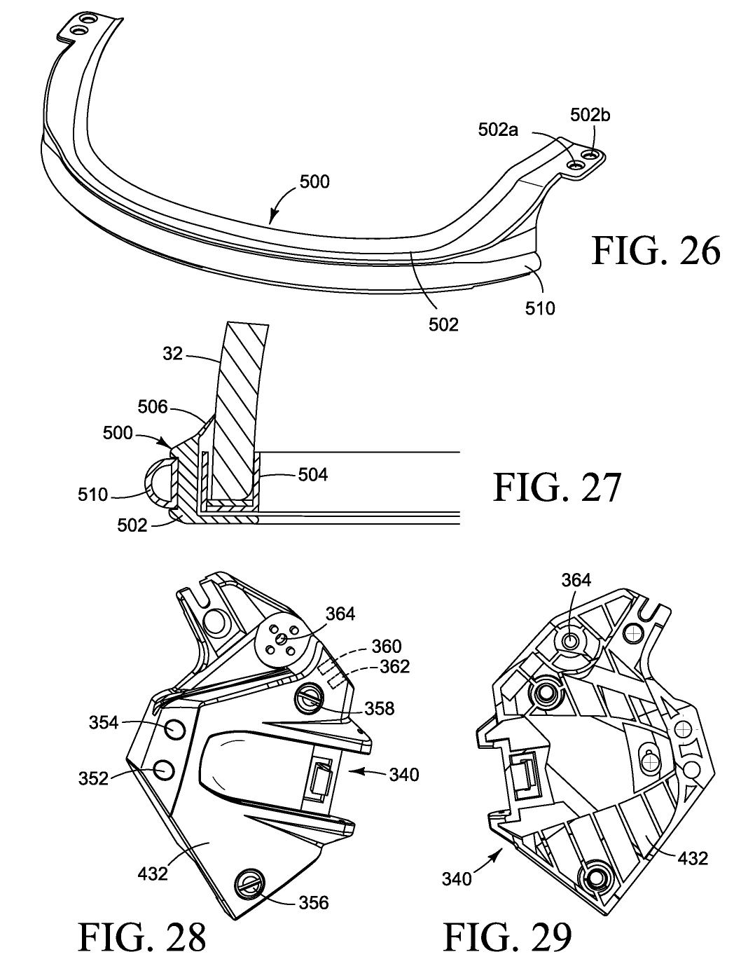

FIGS. 24-27 illustrate the gasket 500 and its attachment of the invention. The gasket 500 includes a somewhat rigid frame 502 that mounts to the helmet brim or rim 504 via the attachment bases 340. The frame includes a flexible lip 506 that seals against the helmet 32. A “D” shaped seal element 510 is adhesively secured to the frame 502. The seal element 510 seals against the shield or visor 44. The frame 502 includes a pair of mounting holes 502a, 502b at each end to secure the frame 502 to each attachment base 340 via the holes 360, 362.

As with the prior attachment base 40, the attachment base 340 includes a plurality of metal sleeves, each one fitting into one of the attachment holes to reinforce the holes and/or to provide threads if needed. The sleeves can be insert molded or overmolded with the body block 432.

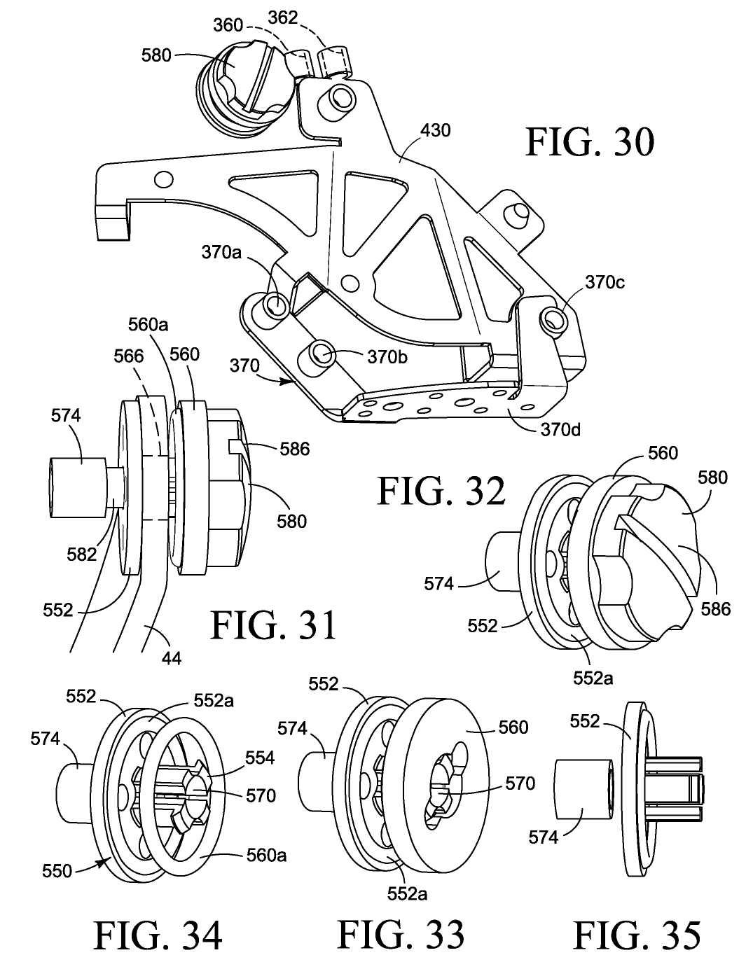

FIGS. 28-35 illustrate an alternate mechanism to pivotally secure the visor 44 to the attachment bases 340. The mechanism includes a base spindle 550 that has a base ring 552 with extending hook elements 554. A clamp ring 560 includes a countersunk hole that receives the hook elements 554 and is engaged thereby to the base spindle 550. The base spindle 550 and the clamp ring 560 are locked together such that they cannot separate or relatively rotate. The hook elements pass through a hole 566 in the visor 44 and the visor is captured between the base spindle 550 and the clamp ring 560. The base ring 552 and the clamp ring 560 each have a circular groove each of which faces the visor 44 and holds an elastomeric o-ring 552a, 560a. A screw channel 570 is present through the clamp ring 560, between the hook element 554 and through the base ring 552. The base ring 552 also includes four spaced apart molding holes wherein the base ring 552 is molded with or overmolded to the block 432. A metal threaded sleeve 574 is also molded into or otherwise integrated to the block 432 into the hole 364 (FIG. 28). A screw 580 having a threaded shank 582 and an enlarged head 586 passes through the channel 570 and is threaded into the sleeve 574. Tightening of the screw compresses the o-rings against the shield 44 and by friction allows the shield to be pivoted to a variety of angles.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

The invention claimed is:

1. An attachment device for a helmet, comprising:
a pair of attachment bases for holding a face shield to the helmet and for attaching to opposite sides of the helmet; and
a strap assembly for extending around a portion of the helmet, for connecting one attachment base to the second attachment base, and for holding the attachment bases against the helmet.

2. The attachment device of claim 1, wherein each attachment base comprises a base plate for engaging the helmet.

3. The attachment device of claim 2, wherein each base plate comprises one or more hooks for engaging an edge of the helmet.

4. The attachment device of claim 1, wherein each attachment base comprises a body block for engaging the strap assembly and for engaging the face shield.

5. The attachment device of claim 2, wherein each attachment base comprises a body block for engaging the strap assembly, for engaging the face shield, and for interchangeably attaching to the base place, the base plate configured to engage a particular type of helmet.

6. The attachment device of claim 1, wherein the strap assembly comprises a pair of tightening straps each for engaging one attachment base.

7. The attachment device of claim 6, wherein the strap assembly comprises a helmet band for connecting the tightening straps and for extending over a portion of the helmet.

8. The attachment device of claim 6, wherein each tightening strap comprises a ratchet toothed strap body for adjusting a tension of the strap against the helmet, and a latch mechanism for securing said selected tension of the strap.

9. The attachment device of claim 8, wherein the latch mechanism is selectively engagable with one of a plurality of teeth on the ratchet toothed strap body.

10. The attachment device of claim 6, wherein each tightening strap comprises a strap tension locking mechanism for securing and releasing a tension of the straps against the helmet, the strap tension locking mechanism comprising an unlocked position wherein the strap tension locking mechanism has an extended length, and a locked position wherein the strap tension locking mechanism has a reduced length to exert tension on the tightening straps.

11. The attachment device of claim 1, wherein each attachment base comprises a mount for attaching the face shield, the mount comprises a resilient detent for holding the rotational position of the face shield, the resilience of the resilient detent may be overcome by applying a predefined amount of force to move the face shield pivotally between an open position, a closed position, and one or more intermediate positions.

12. The attachment device of claim 1, wherein the attachment base comprises one or more attachment holes for attaching auxiliary equipment.

13. The attachment device of claim 12, wherein the attachment holes are threaded.

14. The attachment device of claim 1, wherein the attachment base is also for holding a mandible or mouth guard to the helmet.

15. The attachment device of claim 1, wherein the attachment base comprises a signal element for emitting a signal.

16. The attachment device of claim 15, wherein the signal element emits an inferred signal.

17. The attachment device of claim 1, comprising a visor pivotally attached to said attachment bases and a gasket attached to said attachment bases and sealingly engaged to said visor.

18. A mandible arrangement for a helmet comprising a rigid mandible frame and a ballistic resistant fabric attached to said frame.

19. The arrangement according to claim 18, wherein said fabric hangs below said frame to protect a neck area of the wearer.

20. The arrangement according to claim 19 comprising attachment bases, clamped to said helmet and said mandible arrangement is fastened to said attachment bases.
